(19) United States
(12) Patent Application Publication
(10) Pub. No.: US 2013/0172860 A1
(43) Pub. Date: Jul. 4, 2013

(54) SURGICAL INSTRUMENT IN PARTICULAR FOR PERITONEAL SURGERY

(71) Applicants: Jérôme Szewczyk, Vienne-en-Arthies (FR); Ali Hassan Zarhaee, Saint-Maurice (FR)

(72) Inventors: Jérôme Szewczyk, Vienne-en-Arthies (FR); Ali Hassan Zarhaee, Saint-Maurice (FR)

(73) Assignees: CENTRE NATIONAL DE LA RECHERCHE SCIENTIFIQUE, PARIS (FR); UNIVERSITE PIERRE ET MARIE CURIE (PARIS 6), Paris (FR)

(21) Appl. No.: 13/807,533

(22) PCT Filed: Jun. 29, 2011
(86) PCT No.: PCT/EP11/60871

§ 371 Date: Mar. 12, 2013

(30) Foreign Application Priority Data
Jun. 29, 2010 (FR) 10 55254

(51) Int. Cl. A61B 17/04

(52) U.S. Cl. CPC A61B 17/0469
USPC 606/1

(57) ABSTRACT

A surgical instrument comprising an elongate body which ends with a tool carrier and which is intended to be introduced into the peritoneal cavity by way of a trocar, the body comprising:
an internal tube, which extends along a longitudinal axis of the body and which ends with a bendable stand;
an external tube, which extends around the internal tube, coaxially with respect to the internal tube, and which is mounted so as to rotate about the internal tube; the external tube ending with a bellows, which extends around the stand and which, at its end, bears the tool carrier.

The invention relates to a surgical instrument, in particular for peritoneal surgery. It will be noted that a peritoneal intervention is an intervention carried out within the abdominal cavity.

### TECHNICAL BACKGROUND OF THE INVENTION

Surgical instruments are known that comprise an elongate body and that end with a tool carrier and are intended to be introduced into the peritoneal cavity by way of a trocar.

However, this type of surgical instrument allows only a single rotation of the tool about the longitudinal axis of the surgical instrument. By tilting the instrument, it is also possible, taking the trocar as the bearing, to move the tool inside the abdominal cavity. However, these possibilities of movement are limited and do not permit complex maneuvers, for example suturing. Other instruments have intracorporeal articulations, but these are actuated manually by way of cables and levers, which lead to imprecision and fatigue on the part of a user.

### OBJECT OF THE INVENTION

The object of the invention is to increase the number of movements that can be performed by a tool carried by the tool carrier of a surgical instrument, especially in order to perform complex procedures with this tool, for example suturing.

### BRIEF DESCRIPTION OF THE INVENTION

In order to achieve this object, a surgical instrument is proposed, in particular for peritoneal surgery, comprising an elongate body, which ends with a tool carrier and which is intended to be introduced into the peritoneal cavity by way of a trocar.

According to the invention, the body comprises:
an internal tube, which extends along a longitudinal axis of the body and which ends with a bendable stand;
an external tube, which extends around the internal tube, coaxially with respect to the internal tube, and which is mounted so as to rotate about the internal tube; the external tube ending with a bellows, which extends around the stand and which, at its end, bears the tool carrier.

Thus, the tool carried by the tool carrier of the surgical instrument is made highly mobile by virtue of the following movements:
a translation of the instrument as a whole along its longitudinal axis, which causes a translation of the tool along said longitudinal axis;
a joint rotation of the internal tube and of the external tube, which causes a rotation of the tool about the longitudinal axis of the instrument;
a differential rotation of the tubes, which causes a rotation of the tool about an axis defined by the tool carrier, called the axis proper of the tool;
a bending of the stand, which causes a movement of the tool in the plane of flexion of the stand.

In addition, the possibility of tilting the instrument by using the trocar as a bearing also permits movement of the tool.

The surgical instrument permits in particular the rotation of the tool about its axis proper, which rotation is particularly advantageous since this is a movement required for suturing. In addition, the instrument takes up very little space since the tubes can be of very thin cross section, and since the stand and the bellows, by virtue of being bendable, can be easily inserted into the body of the patient even through a small incision.

### BRIEF DESCRIPTION OF THE DRAWINGS

The invention will be better understood in light of the following description of a specific and non-limiting embodiment of the invention.

Reference will be made to the attached figures, of which:

FIG. 1 is a schematic perspective view of a surgical instrument according to a specific embodiment of the invention, the instrument being arranged on a trocar which is “screwed” onto the abdominal wall of a patient;

FIG. 1a is a perspective and partially cut-away view of a detail of the surgical instrument illustrated in FIG. 1, from a slightly different angle;

FIG. 1b is a slightly enlarged perspective view of a detail of the surgical instrument illustrated in FIG. 1, from a slightly different angle;

FIG. 1c is an enlarged and longitudinal cross-sectional view of the surgical instrument illustrated in FIG. 1;

FIGS. 2a, 2b, 2c, 2d and 2e are partial views of the surgical instrument illustrated in FIG. 1 and depict various possible movements of the surgical instrument;

FIG. 3 is a partial cross-sectional view of the lower part of the surgical instrument according to a second specific embodiment of the invention;

FIG. 4 is a partial cross-sectional view of the lower part of the surgical instrument according to a third specific embodiment of the invention.

### DETAILED DESCRIPTION OF THE INVENTION

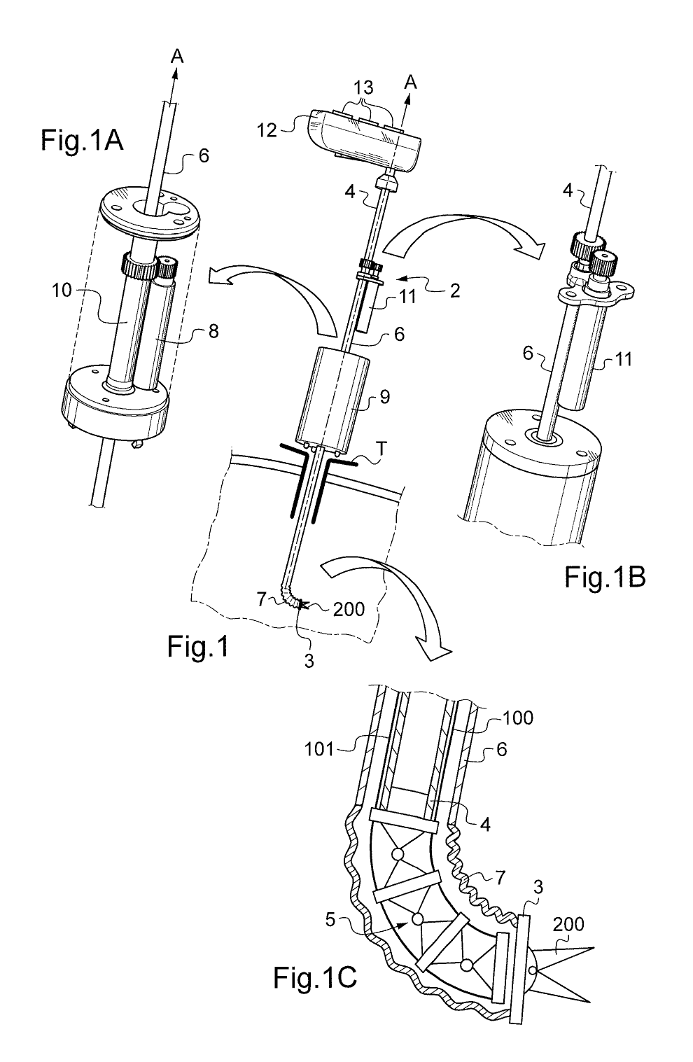

With reference to FIGS. 1, 1a, 1b and 1c, the surgical instrument comprises an elongate body 2, which ends with a tool carrier 3 that carries a tool, in this case a pair of scissors 200.

In the example illustrated, the surgical instrument is used for a peritoneal surgical intervention. To this end, the surgical instrument is introduced into the peritoneal cavity of a patient by way of a trocar T.

According to the invention, the body 2 comprises an internal tube 4, which extends along a longitudinal axis A of the body 2. The internal tube 4 ends with a bendable stand 5. An external tube 6 extends about the internal tube 4, coaxially with respect to the latter, and is able to rotate about the internal tube 4. The external tube 6 ends with a bellows 7, which extends about the stand 5 and which, at its end, bears the tool carrier 3. The two tubes 4, 6 are made of stainless steel, for example. The bellows 7 is made of nickel, for example.

Here, the stand 5 comprises a succession of elements articulated on axes perpendicular to the longitudinal axis A of the body 2 and are parallel to one another.

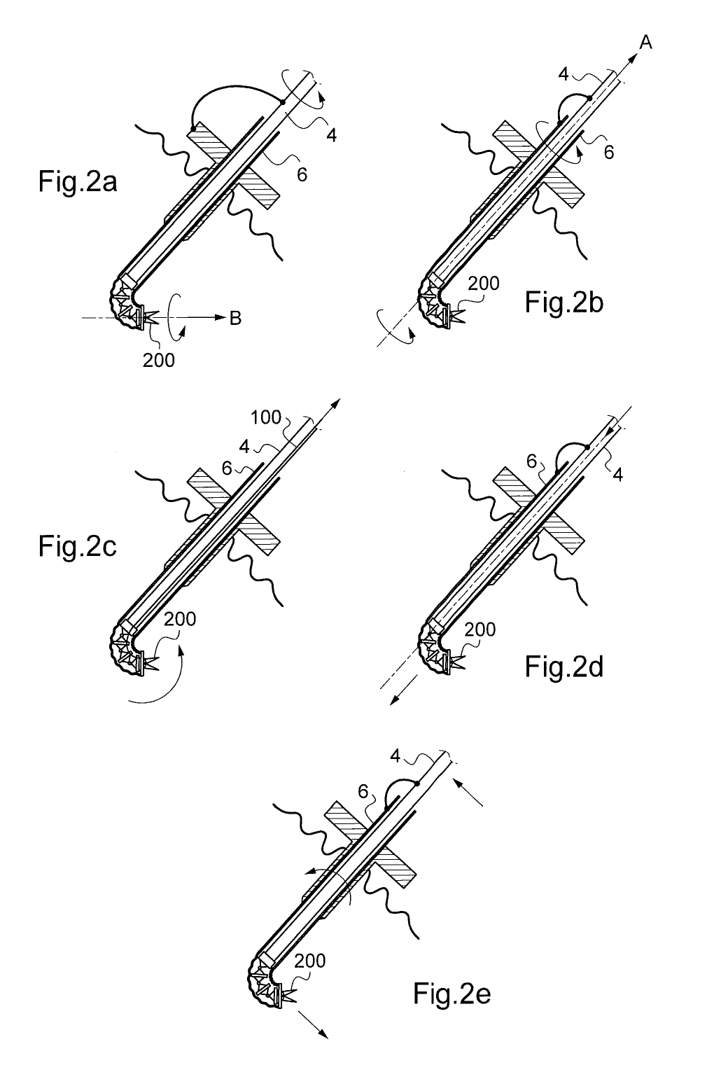

With reference to FIGS. 2a to 2e, the scissors 200 are made highly movable by virtue of the following movements:
By way of the bellows 7, a differential rotation of the two tubes 4, 6 (FIG. 2a) causes a rotation of the scissors 200 about an axis B defined by the tool carrier 3. This axis B is called the axis of rotation proper of the scissors 200;
A joint rotation of the internal tube 4 and of the external tube 6 (FIG. 2b) causes a rotation of the scissors 200 about the longitudinal axis A;
A bending of the stand 5 and bellows 7 causes a movement of the scissors 200 in the plane of flexion of the stand (FIG. 2c);
A translation of the instrument along the longitudinal axis A (FIG. 2d) causes an identical translation of the stand 5 and bellows 7 and therefore of the scissors 200;
A tilting of the body 2 of the tool, taking the trocar T as a bearing (FIG. 2e), causes a translation of the scissors 200 combined with a slight angular movement. This additional translation is advantageous since it takes place along any axis perpendicular to the axis A.

By virtue of the surgical instrument of the invention, a surgeon can perform as many movements with the scissors 200 as he could if he were working directly on the organ on which surgery is to be carried out. The components of the instrument are still of suitable dimensions: the tubes 4, 6 can be of small dimensions, and the unit composed of stand 5 and bellows 7 is deployed only once introduced into the body of the patient.

With reference to FIGS. 1, 1a, 1b and 1c, and according to a preferred embodiment, the surgical instrument comprises three internal actuators for controlling, respectively, the joint rotation of the internal tube 4 and external tube 6, the differential rotation of the internal tube 4 and external tube 6, and the bending of the stand 5. The surgeon then only has to manually perform movements of translation of the instrument through the trocar T and of tilting of the instrument about the trocar T, which are the easiest movements to produce. In particular, the motorization of the actual rotation of the scissors 200 is especially advantageous, since the rotation of the scissors 200 about their own axis is a movement that is very useful in respect of suturing but is generally impossible to do by hand.

To this end, the instrument comprises a first actuator in the form of a first motor 8, which is integral with the trocar T. This avoids the surgeon having to carry the motor 8 when holding the instrument. More precisely, the motor 8 is arranged here in a casing 9 that is designed to be clipped onto the trocar T. The motor 8 is offset from the external tube 6 and drives in rotation a sleeve 10 extending about the external tube 6. Here, the sleeve 10 and the external tube 6 are grooved, such that the rotation of the sleeve 10 drives the external tube 6 in rotation while at the same time permitting a longitudinal movement of the external tube 6 and therefore of the body 2. Preferably, the casing 9 also encloses the tubular component 10.

The instrument likewise comprises a second actuator in the form of a second motor 11, here integral with the external tube 6. In contrast to the first motor 8, which is integral with the trocar T, the second motor 11 is designed to move along with the body 2 during a longitudinal movement of said body 2. The rotor of the second motor 11 rotates the internal tube 4 relative to the external tube 6 about the longitudinal axis A of the instrument, for example by way of a toothed wheel here.

The two motors 8, 11 arranged in this way on the instrument are controlled so as to cause a joint rotation of the two tubes 4, 6 or a differential rotation of the two tubes 4, 6, in such a way as to produce, respectively, a rotation of the scissors 200 about the longitudinal axis A of the instrument and a rotation of the scissors 200 about their own axis of rotation.

Advantageously, the motors 8, 11 are integrated on a part of the instrument that remains outside the body of the patient.

The instrument comprises a third actuator, here for example in the form of two wires 100, 101, which are made of shape-memory alloy and which are attached to the last element of the succession of articulated elements of the stand 5. Thus, a contraction of one or other of the wires causes a bending of the stand 5 and therefore a movement of the scissors 200 in the plane of flexion of the stand 5.

According to a preferred embodiment, the surgical instrument comprises a handle 12, which is here joined to the outer end of the internal tube 4 by way of a connection of the ball-and-socket type (the surgeon only having to manually perform movements of translation and of tilting of the instrument). The ball-and-socket connection thus permits great freedom of movement of the handle 12 with respect to the body 2, making it easier for the surgeon to grip and maneuver the tool.

Here, the handle 12 integrates means for controlling the actuators of the scissors 200. The surgeon can operate the control means, for example by pressing buttons 13 arranged on the handle 12, and thus easily manage the movements of the instrument other than the movements of translation and of tilting.

Preferably, the control means communicate remotely with the two motors 8, 11 and the drive members. The handle 12 is then simply clipped onto the internal tube 4 and can be separated from the body 2. Thus, the handle 12 can be kept, even when the instrument is to be disposed of at the end of an operation.

The invention is not limited to what has just been described and instead covers all variants that fall within the scope defined by the claims.

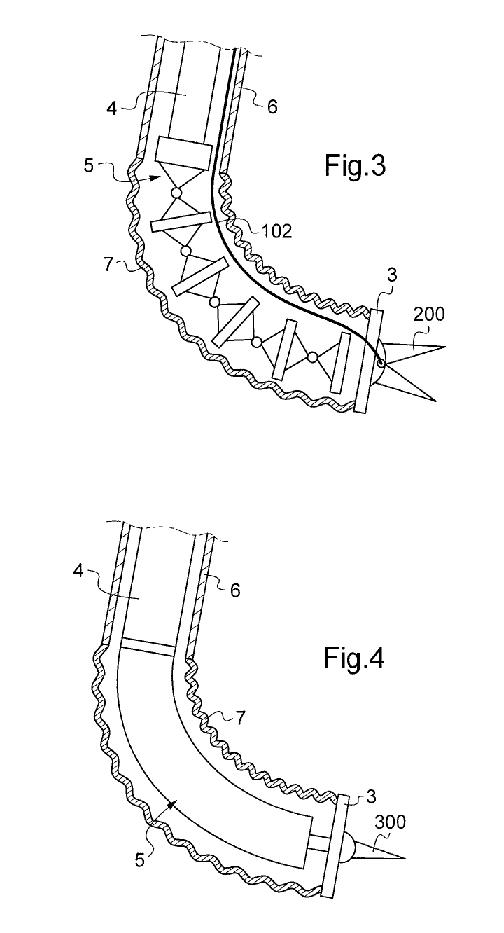

In particular, although the tool carrier here is carried directly by the bellows 7, it is also conceivable that the tool carrier 3 is mounted pivotably on the end of the stand 5. In addition, the instrument will be able to comprise at least one actuator, for example a wire 102 made of shape-memory alloy and extending along the body 2 as far as the tool carrier 3, in order to actuate a tool carried by the tool carrier 3. With reference to FIG. 3, the wire 102 made of shape-memory alloy is able to open and close the scissors 200. In addition, the tool carrier 3 will be able to carry a tool other than the scissors 200, for example a needle 300 for a suturing procedure. In particular, although the second motor 11 is here integral with the external tube 6 and cooperates with the internal tube 4 in order to rotate the internal tube 4 relative to the external tube 6, it will of course be possible for the second motor 11 to be integral with the internal tube 4 and cooperate with the external tube 6 in order to rotate the internal tube 4 relative to the external tube 6.

1. A surgical instrument, in particular for peritoneal surgery, comprising an elongate body which ends with a tool carrier and which is intended to be introduced into the peritoneal cavity by way of a trocar, the body comprising:
an internal tube, which extends along a longitudinal axis of the body and which ends with a bendable stand;
an external tube, which extends around the internal tube, coaxially with respect to the internal tube, and which is mounted so as to rotate about the internal tube; the external tube ending with a bellows, which extends around the stand and which, at its end, bears the tool carrier.

2. The surgical instrument as claimed in claim 1, comprising:
a first motor, which is integral with the trocar and which cooperates with the external tube in order to rotate the external tube relative to the trocar while at the same time permitting a longitudinal movement of the body;
a second motor, which is integral with one of the tubes and which cooperates with the other of the tubes in order to rotate the internal tube relative to the external tube.

3. The surgical instrument as claimed in claim 1, in which the stand comprises a succession of elements articulated on axes perpendicular to the longitudinal axis of the body and are parallel to one another.

4. The surgical instrument as claimed in claim 3, in which the bending of the stand is effected by means of two wires, which are made of shape-memory alloy and which are attached to the last element of the succession of articulated elements of the stand.

5. The surgical instrument as claimed in claim 1, in which the tool carrier is mounted pivotably at the end of the stand.

6. The surgical instrument as claimed in claim 1, comprising at least one actuator for actuating a tool carried by the tool carrier.

7. The surgical instrument as claimed in claim 6, in which the actuator is a wire, which is made of shape-memory alloy and which extends along the body as far as the tool carrier.

8. The surgical instrument as claimed in claim 1, comprising a handle joined to an outer end of the internal tube by a connection of the ball-and-socket type.
